(19) United States
(12) Patent Application Publication
(10) Pub. No.: US 2004/0198828 A1
(43) Pub. Date: Oct. 7, 2004

(54) Combinational use of long-acting and short-acting anti-histamines for ocular allergies

(76) Inventors: Mark B. Abelson, Andover, MA (US); Matthew J. Chapin, Haverhill, MA (US); Paulo J. Gomes, Haverhill, MA (US)

Correspondence Address: Foley Hoag LLP, 155 Seaport Blvd., Boston, MA, 02210-2600, US

(21) Appl. No.: 10/762,201

(22) Filed: Jan. 20, 2004

Related U.S. Application Data
(60) Provisional application No. 60/440,730, filed on Jan. 17, 2003.

(51) Int. Cl. A61K 31/192

(52) U.S. Cl. 514/571

(57) ABSTRACT

The present invention features the combinational use of short-acting anti-histamine agents in combination with long-acting anti-histamine agents to provide rapid, synergistic and long lasting relief towards ocular allergy signs and symptoms.

### CROSS REFERENCE TO RELATED APPLICATIONS

This application claims priority to U.S. Provisional Application Serial No. 60/440,730 filed Jan. 17, 2003.

### BACKGROUND

The eye, particularly the conjunctiva, has a relatively large number of mast cells. When allergens are present, they can bind to immunoglobulins on the surface of these mast cells and trigger the release of cellular contents, known as degranulation. Upon degranulation, mast cell components, including histamine, are released into the environment outside the mast cell. In seasonal or perennial allergic conjunctivitis, these components, particularly histamine are responsible for signs and symptoms associated with allergic responses such as itching, redness, lid swelling, chemosis, tearing, and mucus discharge. In extreme severe chronic cases of ocular allergy (atopic keratoconjunctivitis (AKC) or vernal keratoconjunctivitis (VKC), the sustained reaction produces an inflammatory condition that leads to tissue damage which may result in corneal ulcers.

In the US, an estimated 80 million people experience ocular allergies, according to the American Academy of Allergy, Asthma, and Immunology, and the incidence appears to be on the rise. 90-95% of cases of ocular allergy are either seasonal or perennial allergic conjunctivitis. Allergic symptoms often interfere with everyday activities, such as reading, working on a computer, driving and playing sports. As such, there is a need for pharmaceutical formulations that provide rapid relief from ocular allergic symptoms. Such formulations should also have a long duration of action to eliminate the need for frequent dosing.

### SUMMARY

The invention features novel pharmaceutical compositions of long acting anti-histamine agents and short acting anti-histamine agents that provide synergistic effects towards alleviating the signs and symptoms of ocular allergies. A preferred combination includes an effective concentration of ketotifen and an effective concentration of pheniramine. Another preferred combination includes an effective concentration of azelastine and an effective concentration of antazoline.

A preferred concentration of ketotifen is in the range of about 0.04% to 0.06%. A preferred concentration of pheniramine is in the range of about 0.4% to 0.6%. A preferred concentration of antazoline is in the range of about 0.4% to 0.6% and a preferred concentration of azelastine is in the range of about 0.04% to 0.06%.

The invention also provides for methods of using combinations of long acting anti-histamine agents and short acting anti-histamine agents to treat ocular allergies.

Other features and advantages of the invention will become apparent from the following detailed description and claims.

### BRIEF DESCRIPTION OF THE DRAWINGS

FIGS. 1A and 1B are bar graphs showing mean ocular global redness scores and mean ocular itching scores of subjects treated with placebo or with 0.05% ketotifen (keto) for 15 minutes or 16 hours.

FIGS. 2A and 2B are bar graphs showing mean ocular global redness scores and mean ocular itching scores of subjects treated with placebo or with 0.5% pheniramine (phen) for 15 minutes or 16 hours.

FIGS. 3A and 3B are bar graphs showing mean ocular global redness scores and mean ocular itching scores of subjects treated with placebo or with 0.04% ketotifen (keto) in combination with 0.5% pheniramine (phen) for 16 hours. FIG. 3C is a bar graph comparing median ocular itching scores of subjects treated with placebo or with 0.04% ketotifen (keto) in combination with 0.5% pheniramine (phen) for 16 hours.

FIGS. 4A and 4B are bar graphs showing mean ocular global redness scores and mean ocular itching scores of subjects treated with placebo or with 0.05% ketotifen (keto) in combination with 0.5% pheniramine (phen) for 15 minutes or 16 hours. FIG. 4C is a bar graph comparing median ocular itching scores of subjects treated with placebo or with 0.05% ketotifen (keto) in combination with 0.5% pheniramine (phen) for 16 hours.

FIGS. 5A and 5B are bar graphs showing mean ocular global redness scores and mean ocular itching scores of subjects treated with placebo or with 0.06% ketotifen (keto) in combination with 0.5% pheniramine (phen) for 16 hours. FIG. 5C is a bar graph comparing median ocular itching scores of subjects treated with placebo or with 0.06% ketotifen (keto) in combination with 0.5% pheniramine (phen) for 16 hours.

FIG. 6A is a graph showing mean redness efficacy by treatment with 0.04%, 0.05% or 0.06% ketotifen (keto) in combination with 0.5% pheniramine (phen). FIG. 6B is a graph showing mean itching efficacy by treatment with 0.04%, 0.05% or 0.06% ketotifen (keto)in combination with 0.5% pheniramine (phen).

FIGS. 7A and 7B are bar graphs showing mean ocular global redness scores and mean ocular itching scores of subjects treated with placebo or with 0.05% azelastine for 15 minutes or 4 hours.

FIGS. 8A and 8B are bar graphs showing mean ocular global redness scores and mean ocular itching scores of subjects treated with placebo or with 0.05% azelastine in combination with 0.5% pheniramine for 15 minutes or 4 hours.

FIGS. 9A and 9B are bar graphs showing mean ocular global redness scores and mean ocular itching scores of subjects treated with placebo or with 0.5% antazoline for 15 minutes.

FIGS. 10A and 10B are bar graphs showing mean ocular global redness scores and mean ocular itching scores of subjects treated with placebo or with 0.5% antazoline in combination with 0.05% azelastine for 15 minutes or 16 hours.

### DETAILED DESCRIPTION

### 1. General

The invention is based in part on the surprising discovery that combinational use of short-acting anti-histamine agents such as pheniramine or antazoline, in combination with long-acting anti-histamine agents such as ketotifen or azelastine provide rapid, synergistic and long lasting relief towards ocular allergy signs and symptoms.

### 2. Combinational Use of Long-Acting and Short-Acting Anti-Histamines

The present invention features the combinational use of a long-acting anti-histamine agent and a short-acting anti-histamine agent in the treatment of ocular allergy signs and symptoms such as eye itching, redness, chemosis, lid swelling, tearing and mucus discharge. The term “ocular allergy” refers to any allergic disease of the eye. Examples of such ocular allergies include but are not limited to seasonal/perennial allergic conjunctivitis, vernal keratoconjunctivitis, giant papillary conjunctivitis, perennial allergic conjunctivitis and atopic keratoconjunctivitis. “Seasonal and perennial allergic conjunctivitis” typically occurs in the individual with sensitivities to air borne allergens such as pollens, dust, and animal danders. It is typically seasonal, unlike its year-long cousin, “perennial allergic conjunctivitis”. Both seasonal allergic or perennial allergic conjunctivitis are allergic reactions to materials that do not usually produce such reactions in the normal population. The symptoms of exposure to the material to which the allergic individual is sensitive can include: itchy, running nose with sneezing, and itchy, watery, red, swollen eyes. “Giant papillary conjunctivitis” typically occurs in allergy-prone individuals who wear soft contact lenses. It can occur in individuals who wear other types of contact lenses, but it is more common in soft lens wearers. It occurs as a result of adherence of airborne allergens onto the surface of the contact lens, with eventual development of bumps in the conjunctiva lining the upper eyelid as the allergic/inflammatory response develops over a period of months. The symptoms of this disorder include decreased comfort with contact lens wear, mild itching, excessive contact lens movement, and excessive mucus production. “Vernal keratoconjunctivitis” involves a more complex immunologic/inflammatory process. This disease has major potential for damage to the cornea and loss of vision. The disease affects young people, much more often than older people, is considerably more common in males than in females, and generally occurs in the spring, in temperate climates and is much more common in warmer climates than in temperate or cold climates. It is particularly prevalent in the Middle East and is characterized by the development of very large bumps on the lining of the upper eyelid. Itching is a prominent symptom. Other symptoms and signs include ocular burning, foreign body sensation, excessive tearing, excess mucus production, and blurred vision. “Atopic keratoconjunctivitis” is also a serious allergic eye disease with major blinding potential. It typically occurs in young adults and adults with atopic dermatitis (eczema). Ocular itch is the primary beginning symptom but foreign body sensation, ocular burning, excessive tearing, mucus production, and blurred vision generally eventually occur (http://www.uveitis.org/).

As used herein, the term “anti-histamine agent” may include drugs that counteract the action of histamine. Generally, allergy drugs may include drugs that are more selective for certain sub-types of histamine receptors such as H1 histamine receptor, H2, H3 or H4 receptors. Some anti-histamine agents have less selectivity, and thus more activity across the different histamine receptors, and may even possess activity against other receptors (e.g. cholinergic or adrenergic) which may be involved in regulation of the vasculature. Other anti-histamine agents may additionally act on certain cells, called mast cells, to prevent them from releasing substances that cause the allergic reaction and may also have anti-inflammatory properties.

The term “short-acting anti-histamine agent” may apply to an anti-histamine agent that is typically applied or taken more than once per day or an anti-histamine agent that has varying specificity for histamine receptors and acts to block not just H1 but also to some degree H2, H3, H4 histamine receptors, or other receptors. Such anti-histamine agents may also possess other desirable anti-allergy activities and still have a short duration of action. As used herein “short-acting anti-histamine agent” may include but is not limited to pheniramine (Naphcon-A), chlorpheniramine, dexbrompherniramine, pyrilamine, diphenhydramine (Benadryl), promethazine, hydroxyzine, antazoline, emdastine (Emadine) and pharmaceutically acceptable salts thereof.

The term “long-acting anti-histamine agent” may apply to an anti-histamine agent that is typically applied or taken once or twice per day or an anti-histamine agent that is generally more selective for a particular receptor such as the H1 histamine receptor. Such agents may additionally act on certain cells, called mast cells, to prevent them from releasing substances that cause the allergic reaction and may also have anti-inflammatory properties. As used herein “long-acting anti-histamine agent” refers to but is not limited to ketotifen (Zaditor), loratadine (Claritin), mizolastine, ebastine, fexofenadine (Allegra), Cetrizine (Zyrtec), azelastine, olopatadine (Patanol), desloratadine, carebastine, levoceterizine, astemizole, tecastemizole, epinastine (Elestat), levocabastine (Livostin) and pharmaceutically acceptable salts thereof.

Particular preferred combinations of long-acting anti-histamine agents and short-acting anti-histamine agents reduce ocular redness in about 1 minute, 3 minutes, 5 minutes, 7 minutes, 10 minutes, 15 minutes or 20 minutes. Such combinations may also reduce ocular redness for a duration of 8-10, 10-12, 12-14, 14-16, 16-18, 18-20, 20-22, or 22-24 hours. Particular preferred combinations of long-acting anti-histamine agents and short-acting anti-histamine agents reduce ocular itching in about 1 minute, 3 minutes, 5 minutes, 7 minutes, 10 minutes, 15 minutes or 20 minutes. Such combinations may also reduce ocular itching for a duration of 8-10, 10-12, 12-14, 14-16, 16-18, 18-20, 20-22, or 22-24 hours.

A preferred combination of long-acting anti-histamine agent and short-acting anti-histamine agent is ketotifen or pharmaceutically acceptable salt thereof and pheniramine or pharmaceutically acceptable salt thereof. Yet another preferred combination of long-acting anti-histamine agent and short-acting anti-histamine agent is azelastine or pharmaceutically acceptable salt thereof and antazoline or pharmaceutically acceptable salt thereof.

As used herein, the term “ketotifen” may include a pharmaceutically acceptable salt of ketotifen such as ketotifen fumarate. Particularly preferred concentrations of ketotifen or a pharmaceutically acceptable salt thereof, are in the range of about 0.01 to 0.10%, more preferably in the range of about 0.040 to 0.045%, 0.046 to 0.050%, 0.051 to 0.055% or 0.056 to 0.060%.

As used herein, the term “azelastine” may include a pharmaceutically acceptable salt of azelastine such as azelastine acetate, azelastine guconate, azelastine lactate or azelastine maleate. Particularly preferred concentrations of azelastine or a pharmaceutically acceptable salt thereof, are in the range of about 0.01 to 0.10%, more preferably in the range of about 0.040 to 0.045%, 0.046 to 0.050%, 0.051 to 0.055% or 0.056 to 0.060%, more preferably about 0.05%.

As used herein, the term “pheniramine” may include a pharmaceutically acceptable salt of pheniramine or derivatives of pheniramine such as brompheniramine maleate (Demitane), chlorpheniramine maleate (Chlor-Trimeton), dexbrompheniramine maleate, dexchlorpheniramine maleate (Polaramine), and pheniramine maleate (Naphcon-A). Particularly preferred concentrations of pheniramine or a pharmaceutically acceptable salt thereof, are in the range of about from 0.1 to 1%, more preferably in the range of about 0.40 to 0.45%, 0.46 to 0.50%, 0.51 to 0.55%, or 0.56 to 0.60%, more preferably about 0.5%.

As used herein, the term “antazoline” may include a pharmaceutically acceptable salt of antazoline. Particularly preferred concentrations of antazoline or a pharmaceutically acceptable salt thereof, are in the range of about 0.1 to 1%, more preferably in the range of about 0.40 to 0.45%, 0.46 to 0.50%, 0.51 to 0.55%, or 0.56 to 0.60%, more preferably about 0.5%.

Alternatively, pheniramine or pharmaceutically acceptable salt of pheniramine may be used in combination with another long-acting anti-histamine agent that may include but is not limited to loratadine (Claritin), mizolastine, ebastine, fexofenadine (Allegra), Cetrizine (Zyrtec), olopatadine (Patanol), desloratadine, carebastine, levoceterizine, astemizole, tecastemizole, epinastine (Elestat), emedastine (Emadine) or pharmaceutically acceptable salts thereof.

Alternatively, antazoline or pharmaceutically acceptable salt of antazoline may be used in combination with another long-acting anti-histamine agent that may include but is not limited to ketotifen (Zaditor), loratadine (Claritin), mizolastine, ebastine, fexofenadine (Allegra), Cetrizine (Zyrtec), olopatadine (Patanol), desloratadine, carebastine, levoceterizine, astemizole, tecastemizole, epinastine (Elestat), emedastine (Emadine) and pharmaceutically acceptable salts thereof.

Alternatively, ketotifen or pharmaceutically acceptable salt of ketotifen may be used in combination with another short-acting anti-histamine agent that may include but is not limited to chlorpheniramine, dexbrompheniramine, pyrilamine, diphenhydramine (Benadryl), promethazine, hydroxyzine, antazoline, levocabastine (Livostin) or pharmaceutically acceptable salts thereof.

Alternatively, azelastine or pharmaceutically acceptable salt of azelastine may be used in combination with another short-acting anti-histamine agent that may include but is not limited to chlorpheniramine, dexbrompheniramine, pyrilamine, diphenhydramine (Benadryl), promethazine, hydroxyzine, levocabastine (Livostin) or pharmaceutically acceptable salts thereof.

In one embodiment, an effective concentration of ketotifen or a pharmaceutically acceptable salt thereof may be administered separately from an effective concentration of pheniramine or a pharmaceutically acceptable salt thereof. As used herein, the term “effective concentration” refers to the concentration sufficient to effect a beneficial or desired clinical effect on signs and/or symptoms of ocular allergy upon treatment. An effective concentration of ketotifen may be administered first to the eye surface followed by the administration of an effective concentration of pheniramine. Alternatively, an effective concentration of pheniramine may be administered first to the eye surface followed by the administration of an effective concentration of ketotifen. In another embodiment of the invention, an effective concentration of ketotifen may be administered in combination with an effective concentration of pheniramine at the same time.

In another embodiment, an effective concentration of azelastine or a pharmaceutically acceptable salt thereof may be administered separately from an effective concentration of antazoline or a pharmaceutically acceptable salt thereof. An effective concentration of azelastine may be administered first to the eye surface followed by the administration of an effective concentration of antazoline. Alternatively, an effective concentration of antazoline may be administered first to the eye surface followed by the administration of an effective concentration of azelastine. In another embodiment of the invention, an effective concentration of azelastine may be administered in combination with an effective concentration of antazoline at the same time.

A pharmaceutical composition of the invention may be formulated with any of a variety of carriers including water, mixtures of water and water-miscible solvents, such as C₁- to C₇-alkanols, vegetable oils or mineral oils comprising from 0.5 to 5% by weight hydroxyethyicellulose, ethyl oleate, carboxymethyl-cellulose, polyvinyl-pyrrolidone and other non-toxic water-soluble polymers, in particular for ophthalmic uses, such as, for example, cellulose derivatives, such as methylcellulose, alkali metal salts of carboxymethylcellulose, hydroxymethylcellulose, hydroxyethylcellulose, methylhydroxypropylcellulose and hydroxypropylcellulose, acrylates or methacrylates, such as salts of polyacrylic acid or ethyl acrylate, polyacrylamides, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. Preferred carriers are water, cellulose derivatives, such as methylcellulose, alkali metal salts of carboxymethylcellulose, hydroxymethylcellulose, hydroxyethylcellulose, methylhydroxy-propylcellulose and hydroxypropylcellulose, neutral Carbopol, or mixtures thereof. A highly preferred carrier is water or saline solution. The concentration of the carrier is, typically, from 1 to 100000 times the concentration of the active ingredient. The term “aqueous” typically denotes an aqueous composition wherein the carrier is to an extent of >50%, more preferably >75% and in particular >90% by weight water.

A composition of the invention may include but is not limited to a solution, a suspension, a gel, an ointment, an emulsion and/or a mixture thereof.

Further preference is given to pharmaceutical compositions which are suitable for ocular administration. Therefore such a pharmaceutical composition preferably comprises a pharmaceutically acceptable carrier that include ingredients that meet the prerequisites for ocular tolerability. These further ingredients may include but is not limited to tonicity enhancers, preservatives, solubilizers, non-toxic excipients, demulcents, sequestering agents and pH adjusting agents.

For the adjustment of the pH, preferably to a physiological pH, buffers may especially be useful. The pH of the present solutions should be maintained within the range of 4.0 to 8.0, more preferably about 4.0 to 6.0, more preferably about 6.5 to 7.8. Suitable buffers may be added, such as boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Borate buffers are preferred. Generally, buffers will be used in amounts ranging from about 0.05 to 2.5 percent by weight, and preferably, from 0.1 to 1.5 percent.

Tonicity is adjusted if needed typically by tonicity enhancing agents. Such agents may, for example be of ionic and/or non-ionic type. Examples of ionic tonicity enhancers are alkali metal or earth metal halides, such as, for example, CaCl₂, KBr, KCl, LiCl, Nal, NaBr or NaCl, Na₂SO₄ or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids which is equivalent to a 0.9% solution of sodium chloride or a 2.5% solution of glycerol. An osmolality of about 225 to 400 mOsm/kg is preferred, more preferably 280 to 320 mOsm.

A preservative may typically be selected from a quaternary ammonium compound such as benzalkonium chloride, benzoxonium chloride or the like. Benzalkonium chloride is better described as: N-benzyl-N-(C₈-C₁₈ alkyl)-N,N-dimethylammonium chloride. Examples of preservatives different from quaternary ammonium salts are alkyl-mercury salts of thiosalicylic acid, such as, for example, thiomersal, phenylmercuric nitrate, phenylmercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Germal®II or sorbic acid. Preferred preservatives are quaternary ammonium compounds, in particular benzalkonium chloride or its derivative such as Polyquad (see U.S. Pat. No. 4,407,791), alkyl-mercury salts and parabens. Where appropriate, a sufficient amount of preservative is added to the ophthalmic composition to ensure protection against secondary contaminations during use caused by bacteria and fungi.

In another embodiment, the pharmaceutical compositions of this invention may not include a preservative. Such formulations would be useful for patients who wear contact lenses, or those who use several topical ophthalmic drops and/or those with an already compromised ocular surface (e.g. dry eye) wherein limiting exposure to a preservative may be more desirable.

A pharmaceutical composition may additionally require the presence of a solubilizer, in particular if the active or the inactive ingredients tends to form a suspension or an emulsion. A solubilizer suitable for an above concerned composition is for example selected from the group consisting of tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. A specific example of an especially preferred solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient.

An above pharmaceutical composition may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. Other excipients that may be used if desired are listed below but they are not intended to limit in any way the scope of the possible excipients. The amount and type of excipient added is in accordance with the particular requirements and is generally in the range of from approximately 0.0001 to approximately 90% by weight.

The pharmaceutical composition of the invention may comprise further demulcents such as povidone (see U.S. Pat. No. 6,274,626), a Category I demulcent in the OTC Ophthahnic Drug Products Monograph of the USFDA. Polyvinylpyrrolidone (PVP) is a linear homopolymer or copolymer comprising at least about 80%, preferably at least about 90% of repeat units derived from 1-vinyl-2-pyrrolidone monomers, the polymer more preferably comprising at least about 95% or essentially all of such repeat units, the remainder selected from polymerization-compatible monomers, preferably neutral monomers, such as alkenes or acrylates. Other synonyms for PVP include povidone, polyvidone, 1-vinyl-2-pyrolidinone, and 1-ethenyl-2-pyrolionone (CAS registry number 9003-39-8). PVP has a weight average molecular weight of about 10,000 to 250,000, preferably 30,000 to 100,000. Such materials are sold by various companies, including ISP Technologies, Inc. under the trademark PLASDONE™ K-29/32, BASF under the trademark KOLLIDON™ for USP grade PVP, for example KOLLIDON™ K-30 or K-90 (BASF Corporation, NV Division, 3000 Continental, Mount Olive, N.J. 07628-1234, USA). It is to be understood, however, that the pharmaceutical compositions of this invention is not limited to any specific PVP and that any equivalent PVP of acceptable purity for ophthalmic use, preferably pharmaceutical grade, may be used. PVP also acts as a water-soluble viscosity builder. Optionally, additional viscosity builders or demulcents may be employed in the present composition, for example, cellulose derivatives, glycerin, and the like. Such viscosity builders or demulcents may be employed in a total amount ranging from about 0.01 to about 5.0 weight percent or less. Suitably, the viscosity of the final formulation is 10 cps to 50 cps. In the present compositions, povidone may be suitably present in a total amount of 0.1 to 5.0% by weight, preferably 0.5 to 2.0 percent by weight of the composition.

Typically, compositions for treating the symptoms of allergy that have been on the market are not recommended for use with lenses in place. As previously described, pharmaceutical compositions of the present invention may be used with or without the contact lenses in place, so that contact lenses do not have to be removed. In addition to buffering agents, in some instances it may be desirable to include sequestering agents in the present solutions in order to bind metal ions that might otherwise react with the lens and/or protein deposits and collect on the lens. Ethylene-diaminetetraacetic acid (EDTA) and its salts (disodium) are preferred examples. They are usually added in amounts ranging from about 0.01 to about 0.2 weight percent.

The solutions of the invention may be administered daily from 1, 2, 3, 4, or 5 drops depending on the severity of the allergy reactions. Compositions according to the present invention can be applied by instilling about 1, 2, 3 4, or 5 drops in the affected eye(s) or in an effective amount as needed for rapid and long lasting relief of symptoms due ocular allergies. An “effective amount” is an amount sufficient to effect a beneficial or desired clinical result upon treatment. An effective amount can be administered to a patient in one or more doses. In terms of treatment, an effective amount is an amount that is sufficient to decrease or ablate ocular allergy signs and/or symptoms in a subject. Several factors are typically taken into account when determining an appropriate dosage to achieve an effective amount. These factors include the form and effective concentration of the pharmaceutical composition being administered.

Compositions of the present invention may also include artificial tears and can be used, as needed, for the temporary relief of eye irritation or discomfort. As used herein the term “artificial tears” may include mixtures of fluid compounds to substitute for naturally produced tears. For example, many people suffer from eye conditions in which the eye's tear system fails to provide adequate tear volume or tear film stability necessary to remove irritating environmental contaminants such as dust, pollen, or the like. In persons suffering from the symptoms of dryness, the film on the eye tends to become discontinuous. Because of their emollient and lubricating effect, artificial tears can be used to soothe the eye and may be part of the formulation in this invention

All publications and patents mentioned herein are hereby incorporated by reference in their entirety as if each individual publication or patent was specifically and individually indicated to be incorporated by reference. In case of conflict, the present application, including any definitions herein, will control.

The invention now being generally described, it will be more readily understood by reference to the following examples, which are included merely for purposes of illustration of certain aspects and embodiments of the present invention, and are not intended to limit the invention.

### EXEMPLIFICATION

### EXAMPLE 1

### Methods of Conjunctival Allergen Challenge (CAC)

These following studies were performed using the standard conjunctival allergen challenge model as described in Abelson et al., Arch Ophthalmol. 108(1): 84-88 (1990), and Abelson et al., Curr Allergy Asthma Rep. 3(4): 363-8 (2003). Each experiment was a double-masked, contralateral placebo controlled, single center, onset of action and duration of action allergen challenge study.

A baseline ocular exam was performed to rule out any subjects with any ocular surface anomaly that would interfere with evaluations. A bilateral conjunctival allergen challenge was performed with the allergen to which the subject was known to be allergic to as evidenced by a skin test or previous conjunctival allergen challenge.

Subjects were administered one drop of masked study medication in each eye as described in the below tables (see Table 1-3). A period of at least 1 week was left between experiments on each single subject. At the specified timepoints, subjects were challenged bilaterally with one drop of allergen solution (allergen used were birch, cat hair, cat dander) at a concentration that was known to historically elicit a positive response (Abelson et al., Arch Ophthalmol. 108(1): 84-88 (1990)). Phosphate buffered saline (PBS) was used as placebo. A historic positive response to the challenge was defined as at least a +2.0 itching and +2.0 redness on scales 0-4, with 0 being none and 4 being severe.

Subjects were asked to grade their ocular itching on standardized scales described in Abelson et al., Arch Ophthalmol. 108(1): 84-88 (1990), scoring was performed from a scale of 0-4 at 3, 5, and 7 minutes post-challenge. Redness was graded by a masked observer on standardized scales 0-4 at 10, 15, and 20 minutes post-challenge. Digital photographs were taken of each eye at the 15 minute timepoint. If needed, subjects were administered one drop of a currently marketed topical anti-allergic medication (Naphcon A™: pheniramine maleate 0.3%/naphthazoline hydrochloride 0.025% ophthalmic solution) to relieve any immediate discomfort at the end of the visit.

### EXAMPLE 2

### Statistical Analysis

Data from all subjects who completed any post-challenge examination was evaluable for that experiment and treatment. Mean itching and redness scores were compared between the treated eye and placebo eye to determine an efficacy score. Data was summarized in appropriate tables.

TABLE 1
Mean Differences (Active-Placebo)-Ketotifen (keto)
0.05% + Pheniramine 0.5%

| | | Timepoint | Timepoint | Timepoint |
|---|---|---|---|---|
| DRUG | Evaluation | 1 | 2 | 3 |
| Keto 0.05% + | 15 Min Itching | −2.67 | −2.67 | −2.5 |
| Pheniramine | 15 Min Redness | −2.33 | −2.67 | −2.83 |
| 0.5% (N = 3) | 16 hr Itching | −1.33 | −2.00 | −1.83 |
| | 16 hr Redness | −0.67 | −0.67 | −0.83 |
| Keto 0.05% | 15 Min Itching | −2.00 | −2.33 | −2.00 |
| (N = 3, 2) | 15 Min Redness | −1.50 | −1.17 | −1.50 |
| | 16 hr Itching | 0.75 | 0.25 | 0.25 |
| | 16 hr Redness | 0.5 | 0.5 | 0.25 |
| Pheniramine | 15 Min Itching | −2.67 | −2.33 | −2.17 |
| 0.5% (N = 3; 2) | 15 Min Redness | −1.67 | −1.83 | −1.67 |
| | 16 hr Itching | −0.25 | 0 | 0 |
| | 16 hr Redness | 0.25 | 0.25 | 0.25 |

TABLE 2
Mean Differences (Active-Placebo)-Between
Different Concentrations of Ketotifen (keto)
X % + Pheniramine 0.5%

| | | | | Timepoint |
|---|---|---|---|---|
| DRUG | Evaluation | Timepoint 1 | Timepoint 2 | 3 |
| Keto 0.05% + | 16 hr Itching | −1.33 | −2.00 | −1.83 |
| Pheniramine | 16 hr Redness | −0.67 | −0.67 | −0.83 |
| 0.5% (N = 3) | | | | |
| Keto 0.04% + | 16 hr Itching | −0.67 | −1.00 | −0.83 |
| Pheniramine | 16 hr Redness | −0.5 | −0.67 | −0.67 |
| 0.5% (N = 3) | | | | |
| Keto 0.06% + | 16 hr Itching | −1.5 | −1.83 | −2.17 |
| Pheniramine | 16 hr Redness | −0.17 | −0.17 | 0.17 |
| 0.5% (N = 3) | | | | |

TABLE 3
Mean Differences (Active-Placebo)-Azelastine
0.05% + Antazoline 0.5%,
Azelastine 0.05% + Pheniramine 0.5%.

| | | Timepoint | Timepoint | Timepoint |
|---|---|---|---|---|
| DRUG | Evaluation | 1 | 2 | 3 |
| Azelastine | 15 Min Itching | −2.50 | −2.33 | −1.67 |
| 0.05% + | 15 Min Redness | −0.33 | 0 | 0 |
| Antazoline | 16 hr Itching | −0.5 | −0.75 | −0.5 |
| 0.5% (N = 3) | 16 hr Redness | 0.25 | 0.5 | 0.5 |
| Azelastine | 15 Min Itching | −2.5 | −2.5 | −2.5 |
| 0.05% (N = 1 | 15 Min Redness | 0 | 0 | 0 |
| for 15 min and | 4 hr Itching | −0.5 | −1.0 | −1.0 |
| N = 3 for | 4 hr Redness | +0.5 | +1.0 | +1.0 |
| duration) | | | | |
| Antazoline 0.5% | 15 Min Itching | −2.33 | −2.83 | −2.5 |
| (N = 3) | 15 Min Redness | −0.83 | −0.83 | −1.00 |
| Azelastine | 15 Min Itching | −1.0 | −1.0 | −0.67 |
| 0.05% + | 15 Min Redness | −0.88 | −1.17 | −1.17 |
| Pheniramine | 4 hr Itching | −2.33 | −2.67 | −2.5 |
| 0.5% (N = 3) | 4 hr Redness | −1.0 | −1.33 | −1.0 |

Results

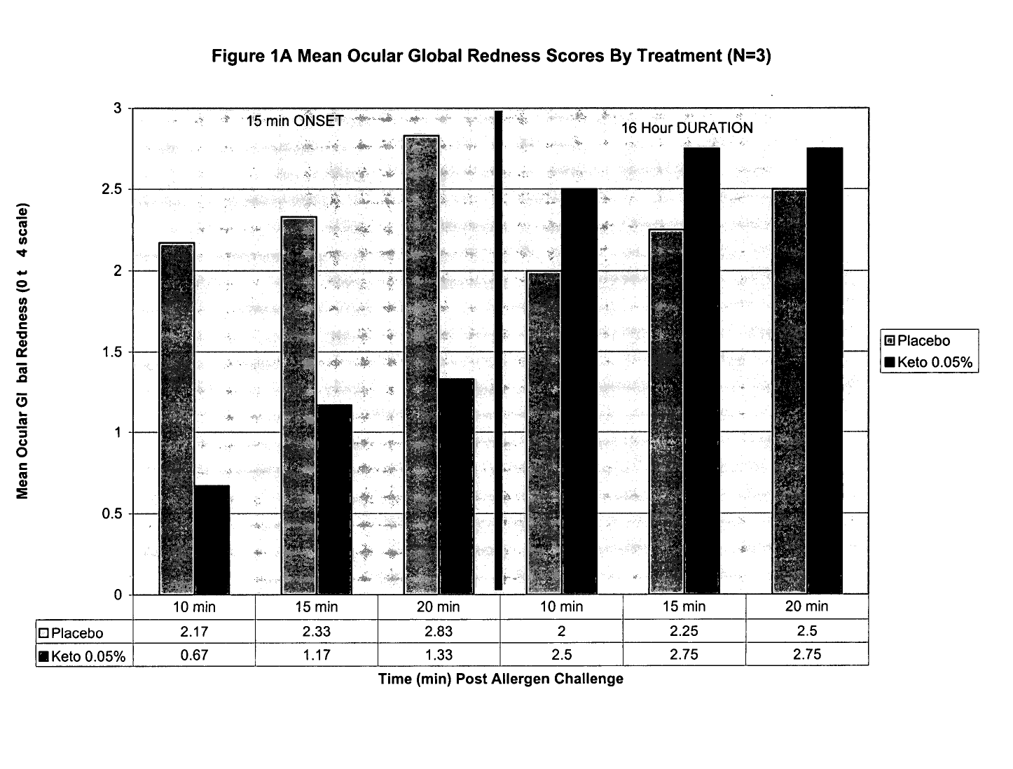

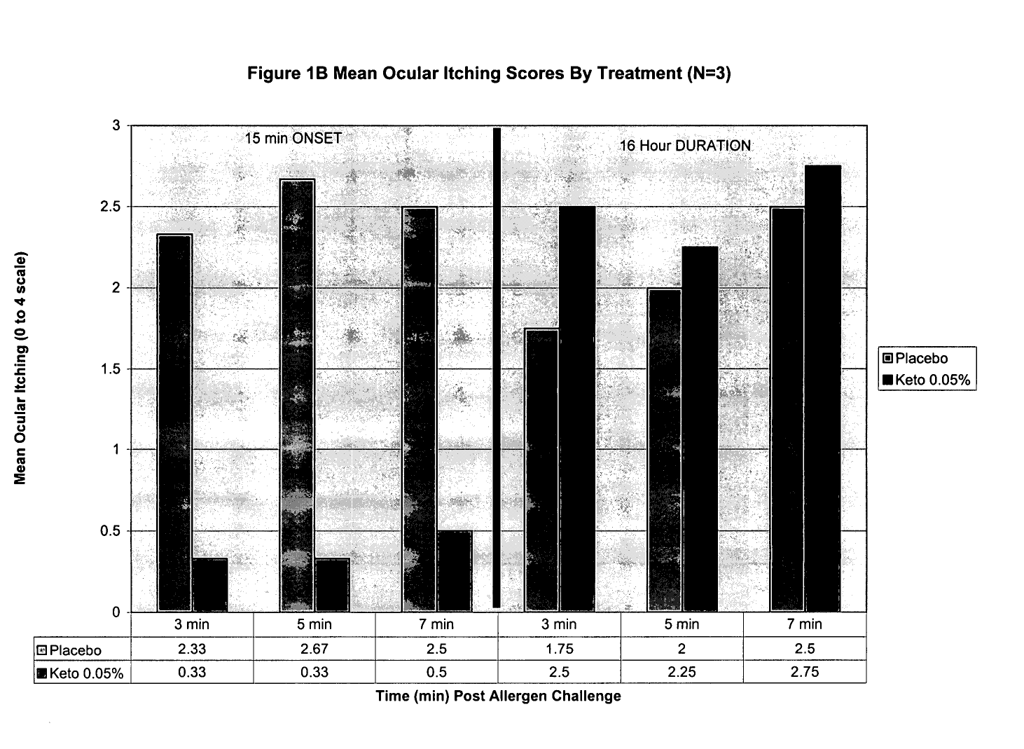

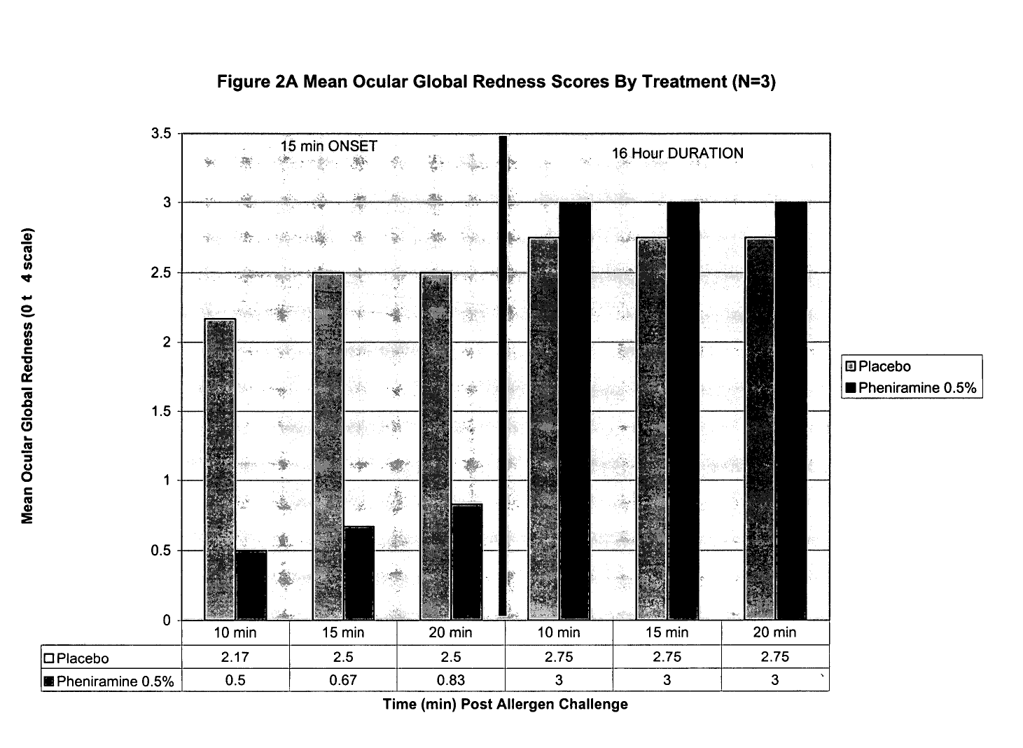

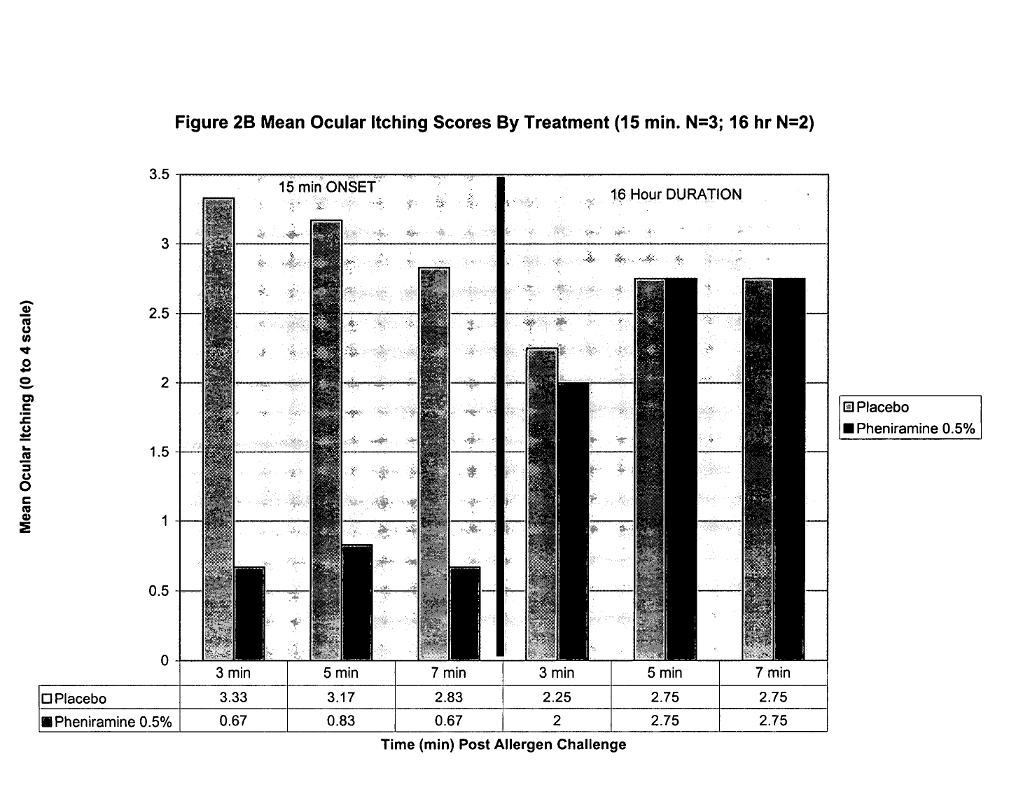

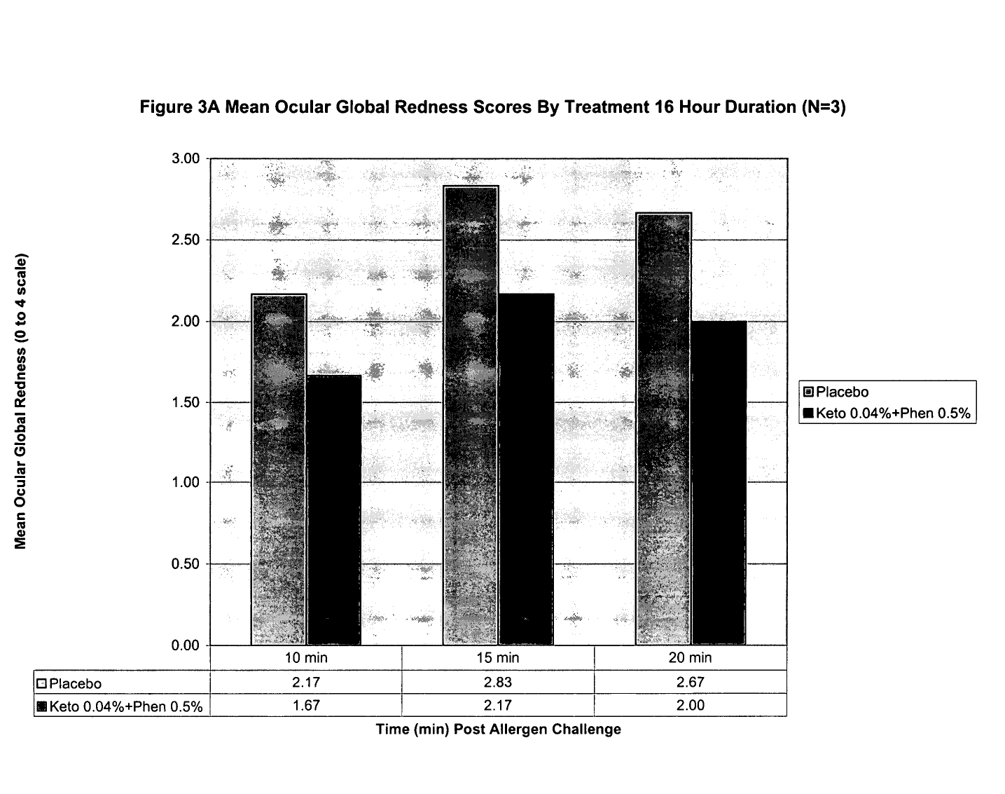

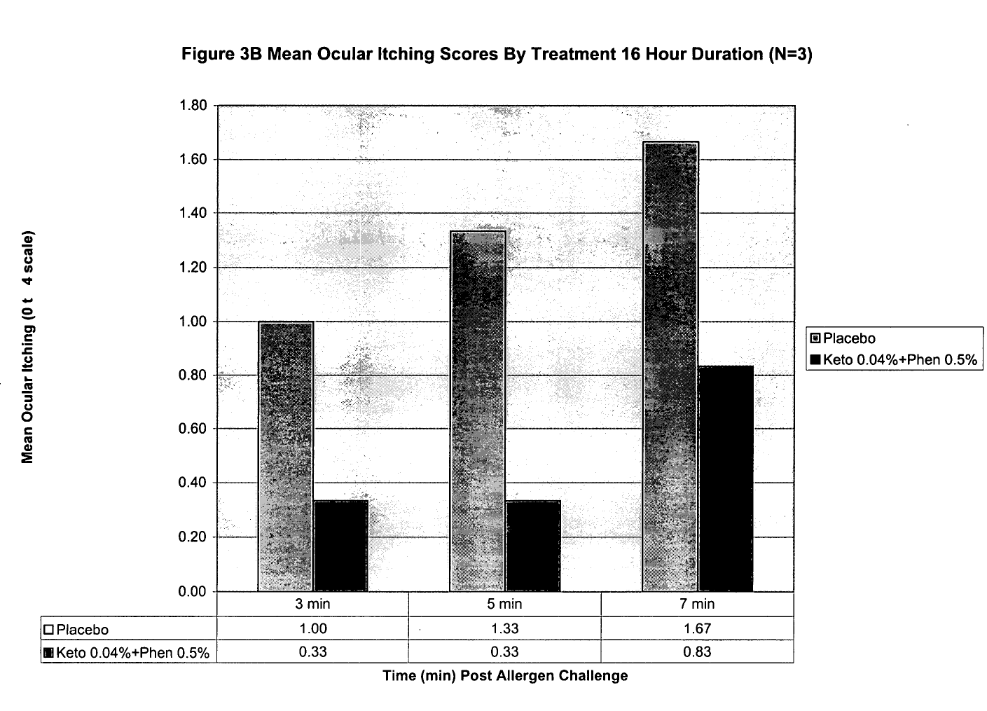

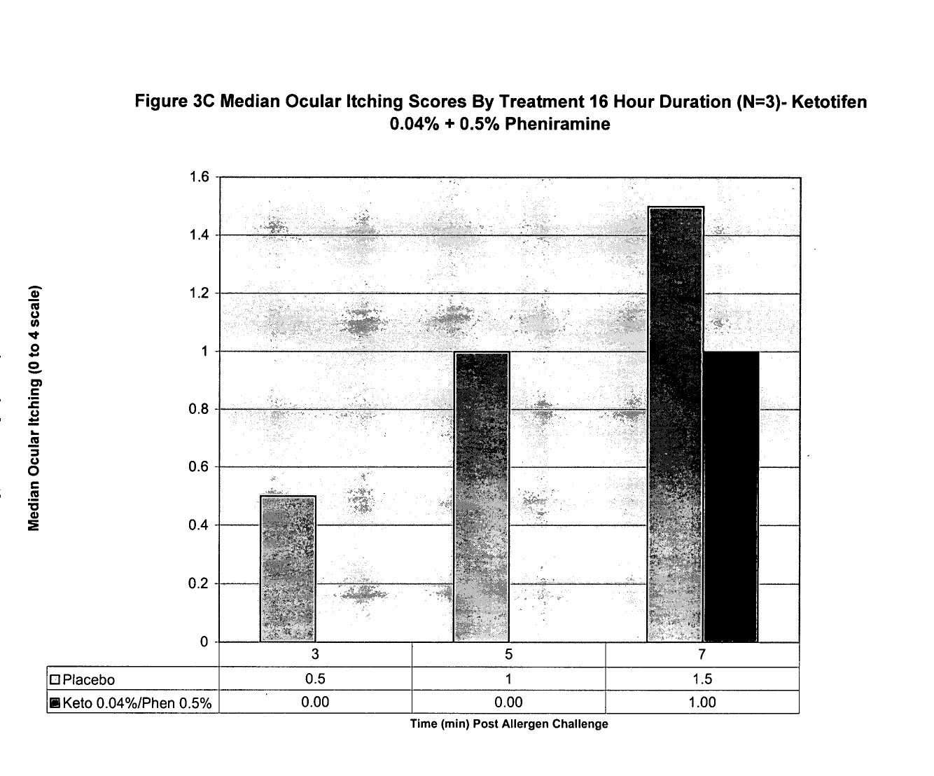

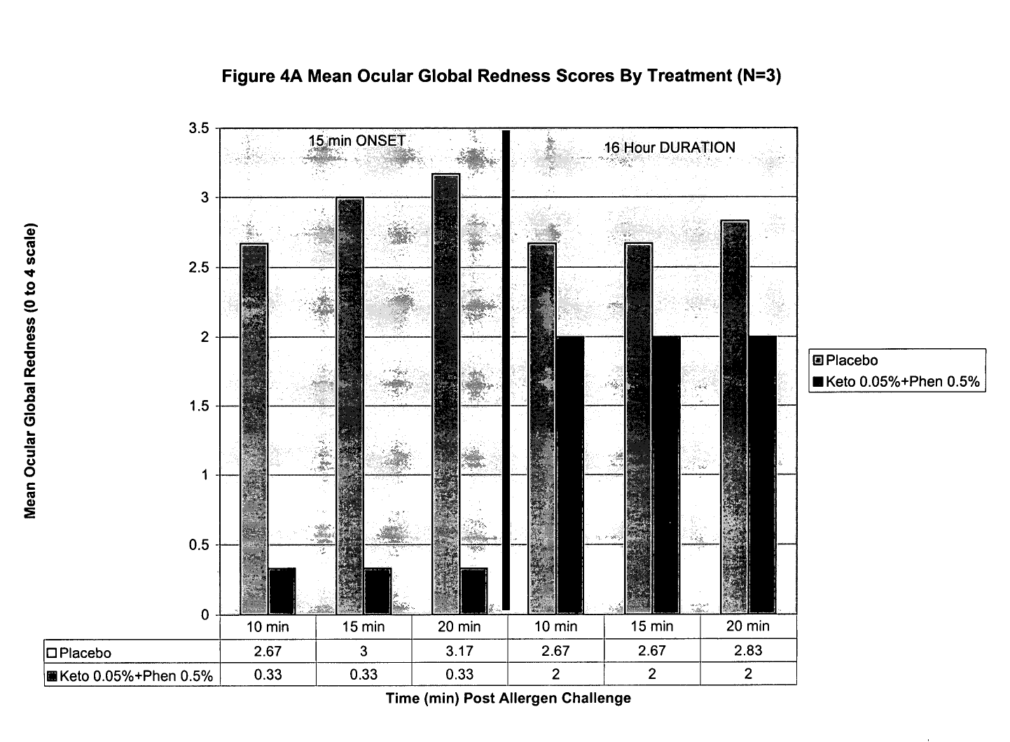

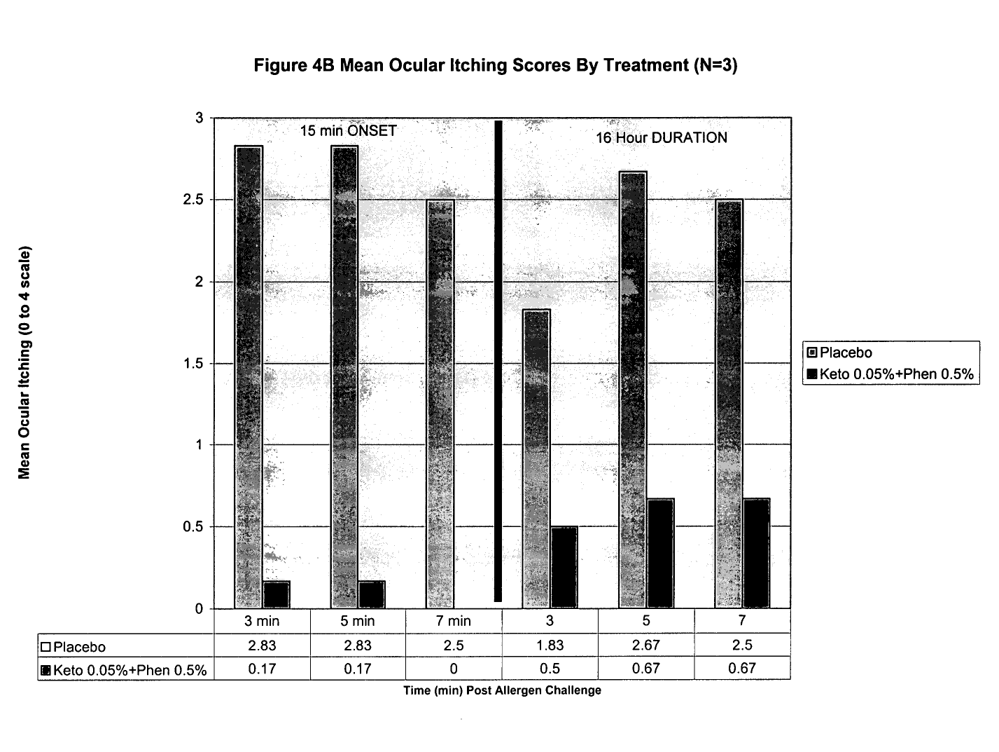

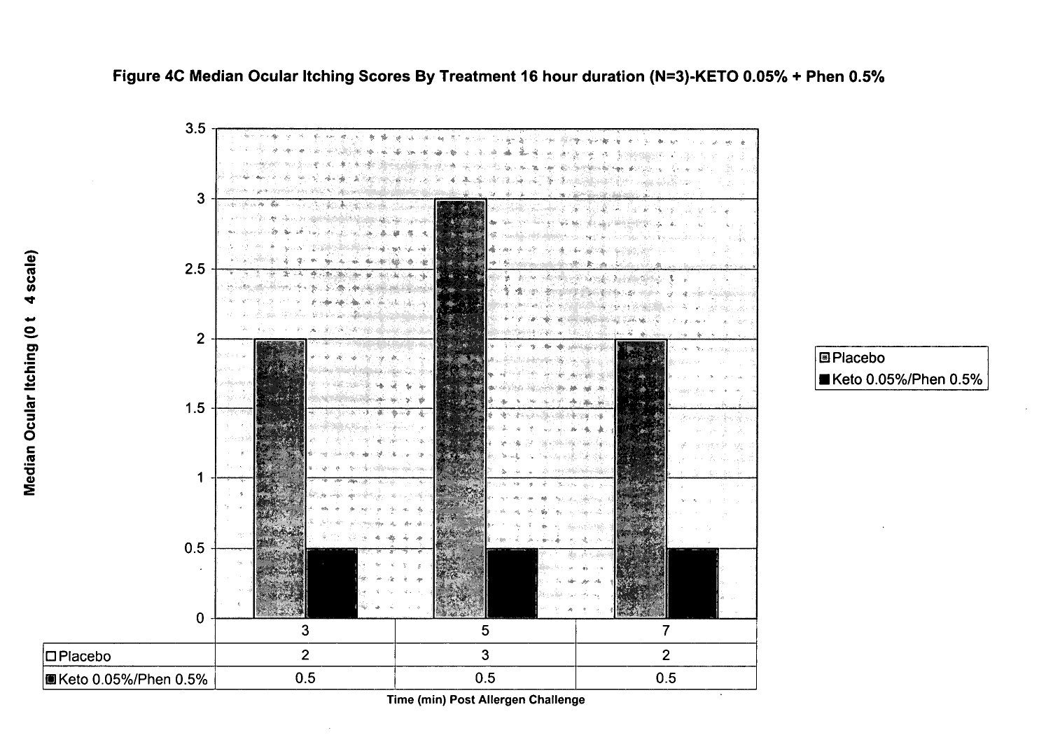

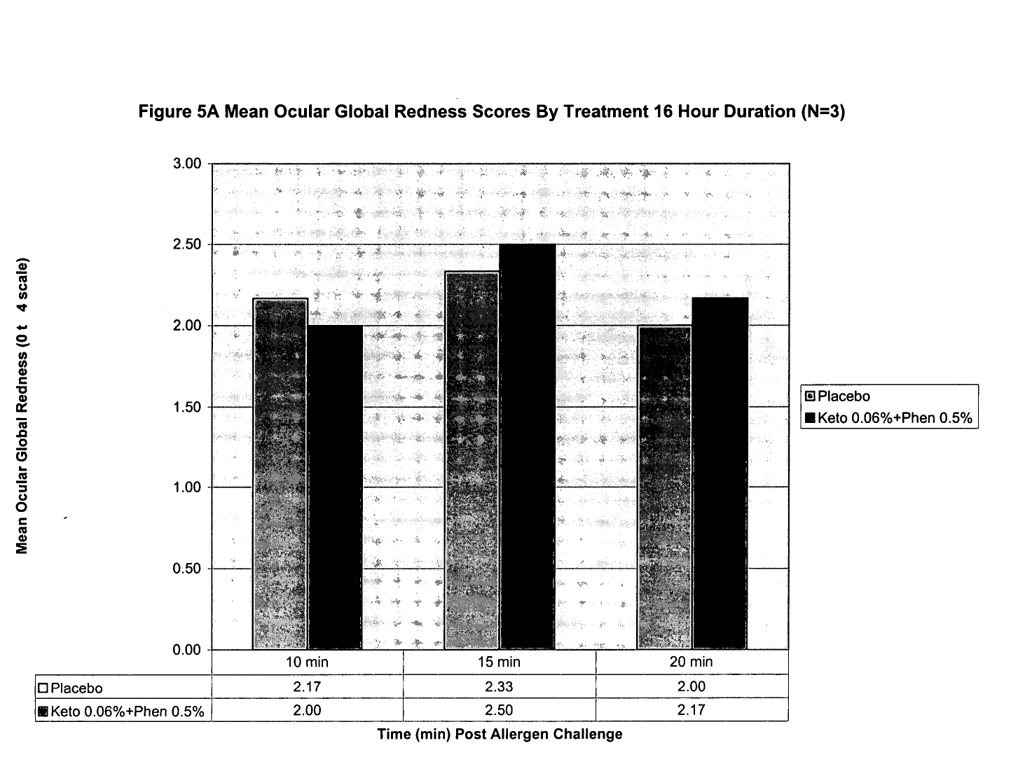

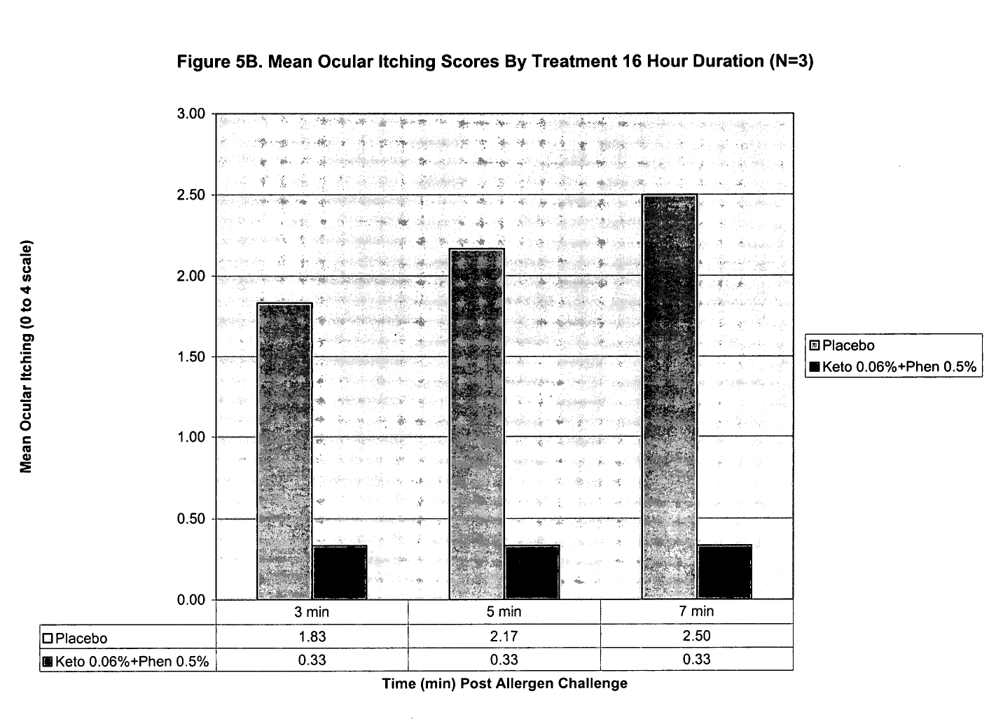

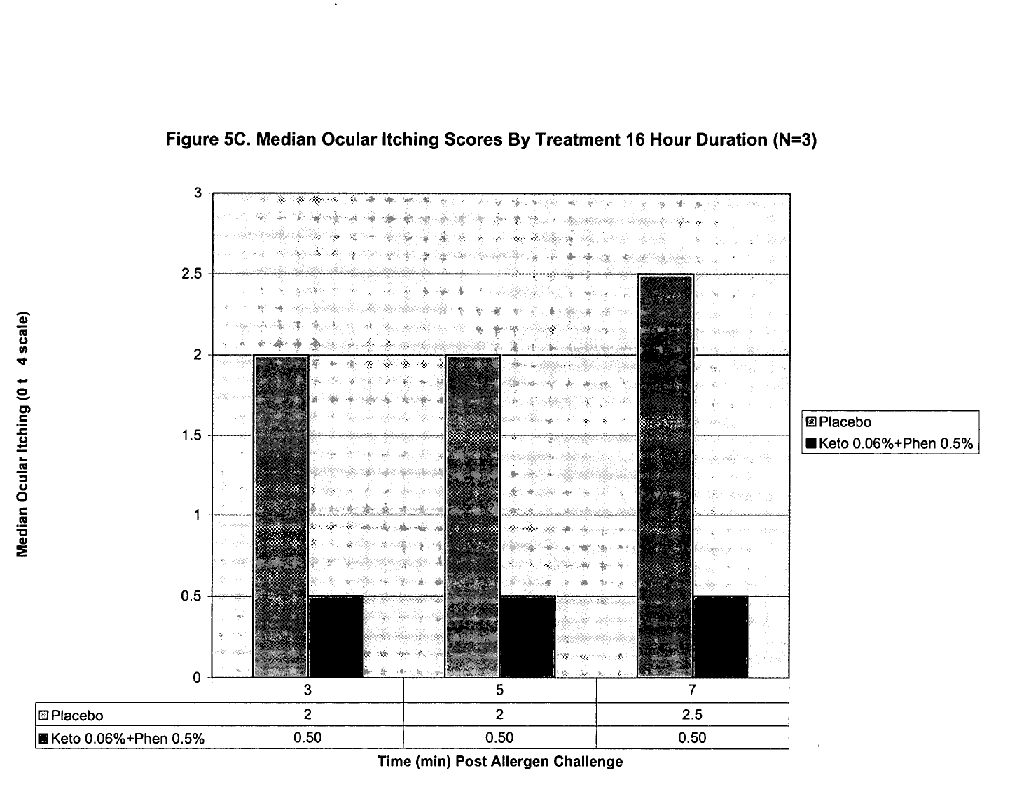

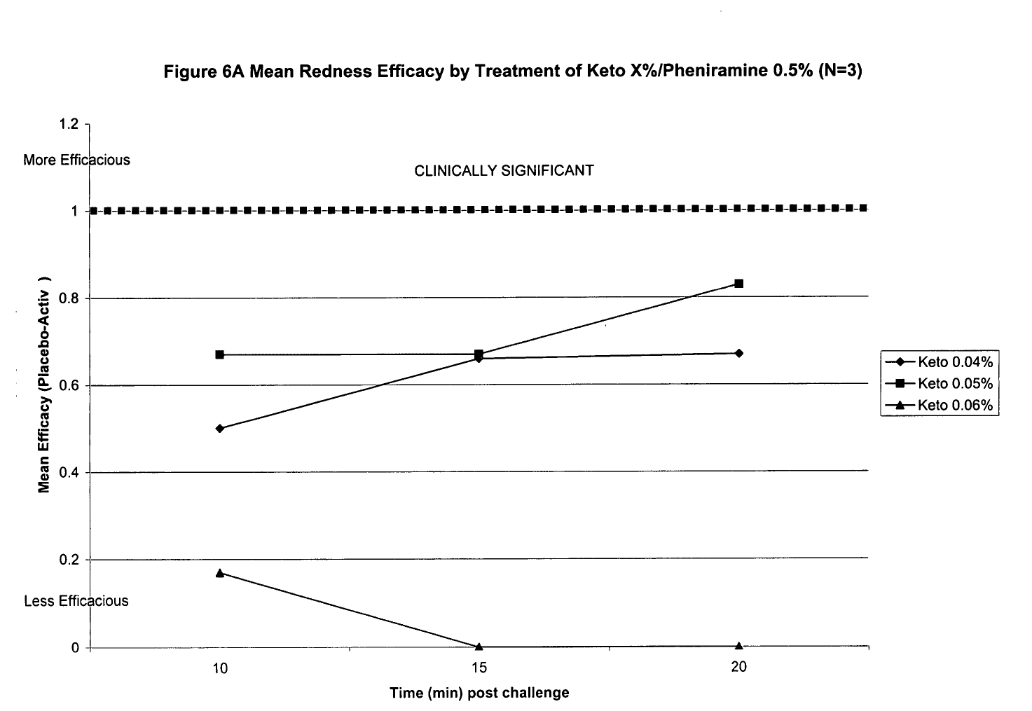

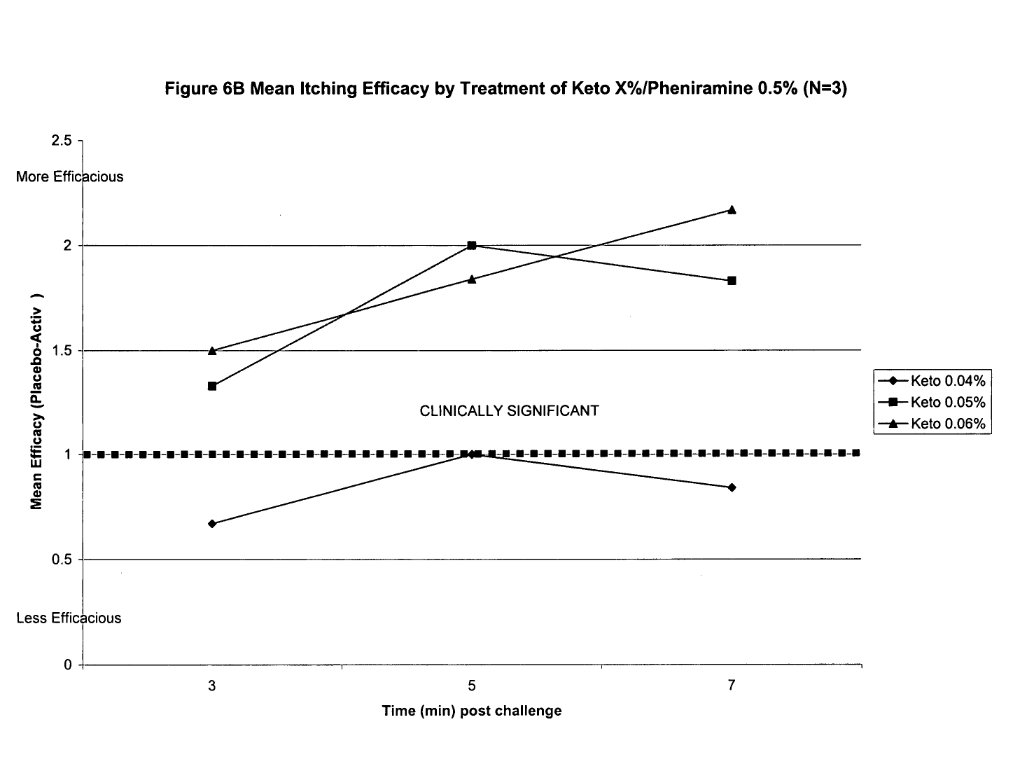

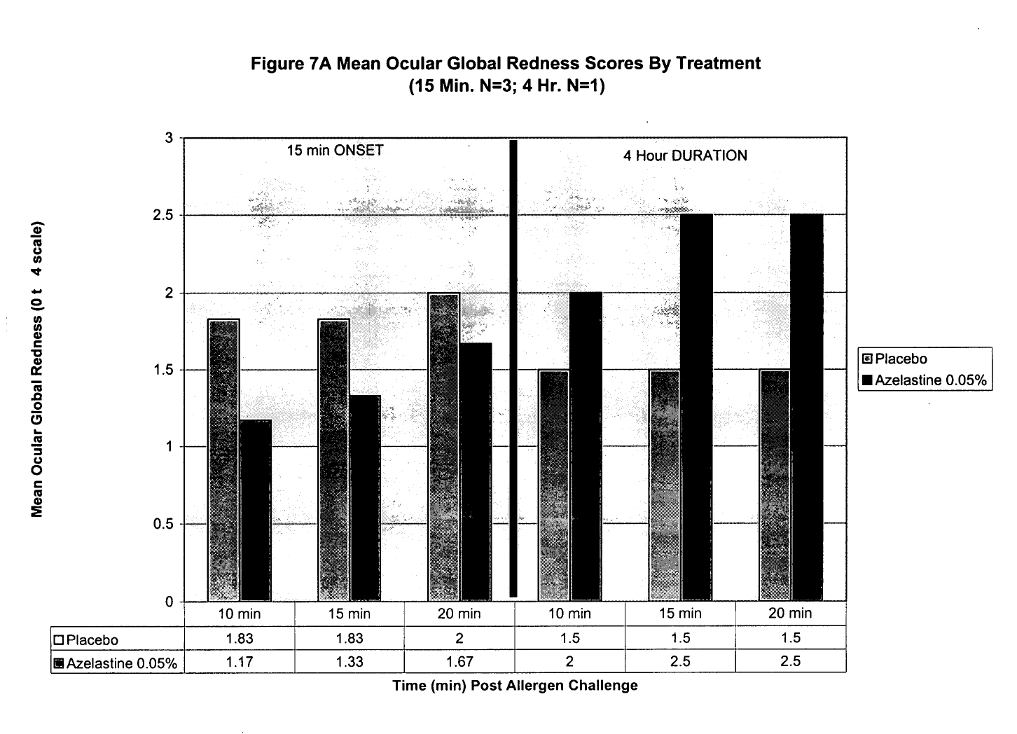

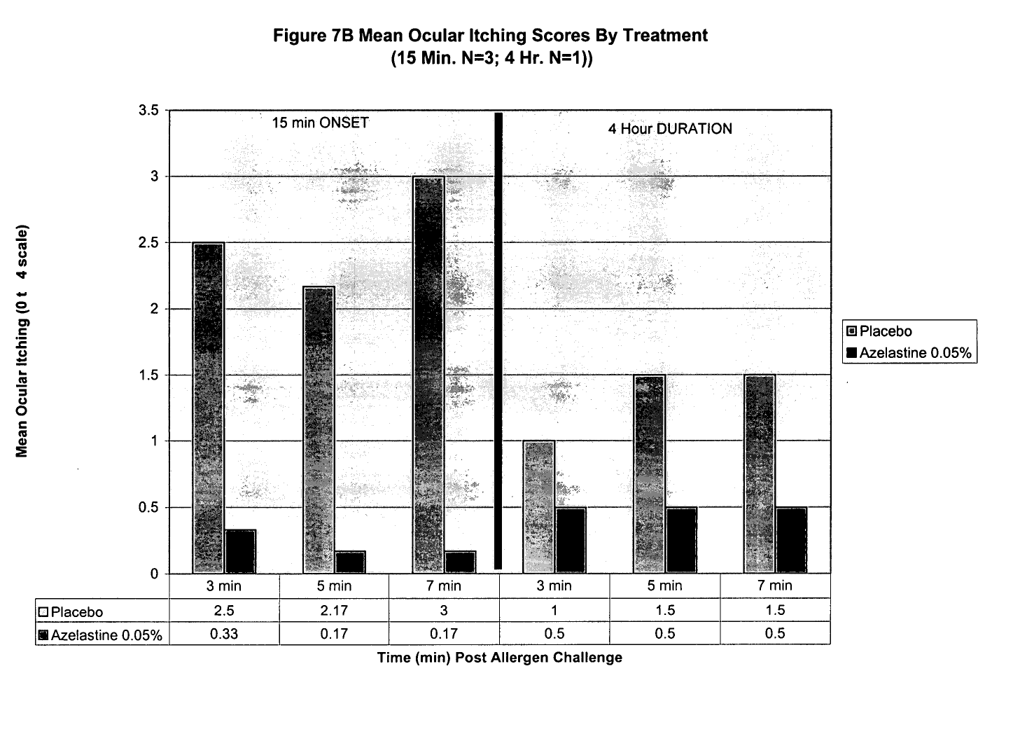

The combinational use of 0.05% ketotifen and 0.5% pheniramine has greater efficacy when compared to the 0.05% ketotifen alone and 0.5% pheniramine alone in relieving both itching and redness at both onset and duration (see FIGS. 1, 2, 4, 6). For itching higher concentrations of ketotifen in the combination product are more effective in a dose dependent manner. This is not seen for redness where the optimal concentration is 0.05% (see FIG. 6).

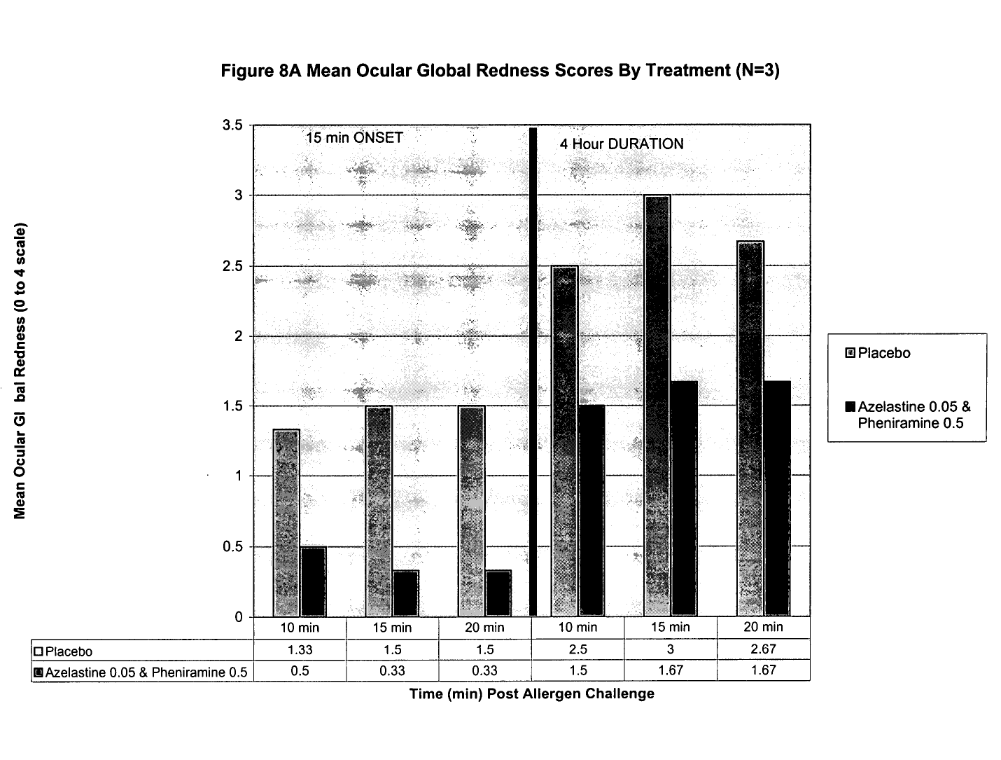

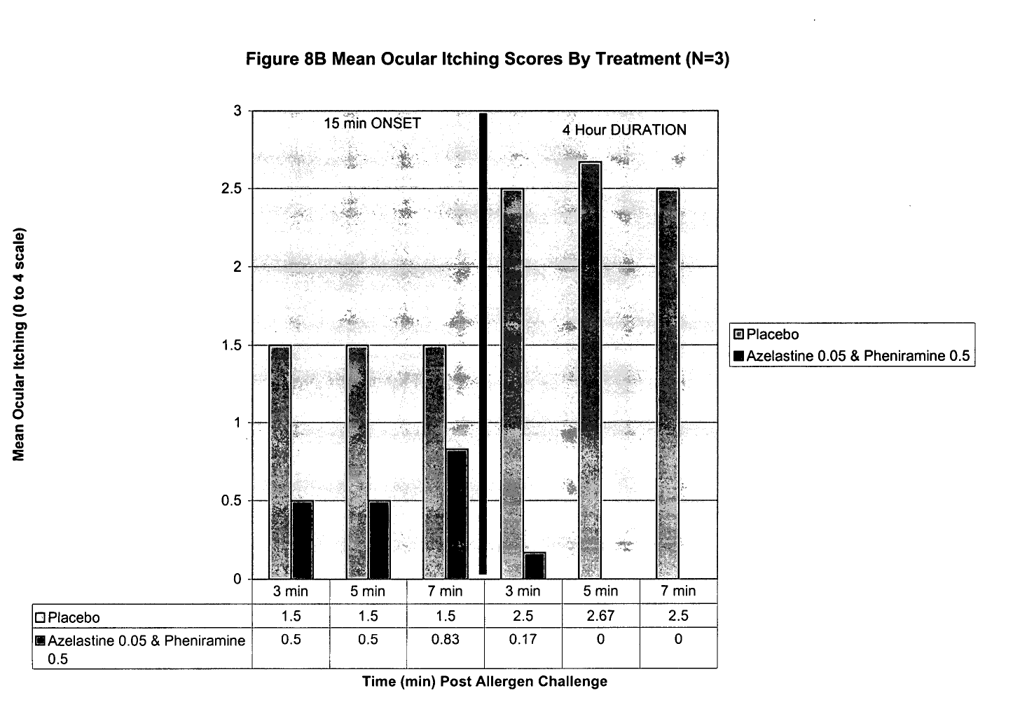

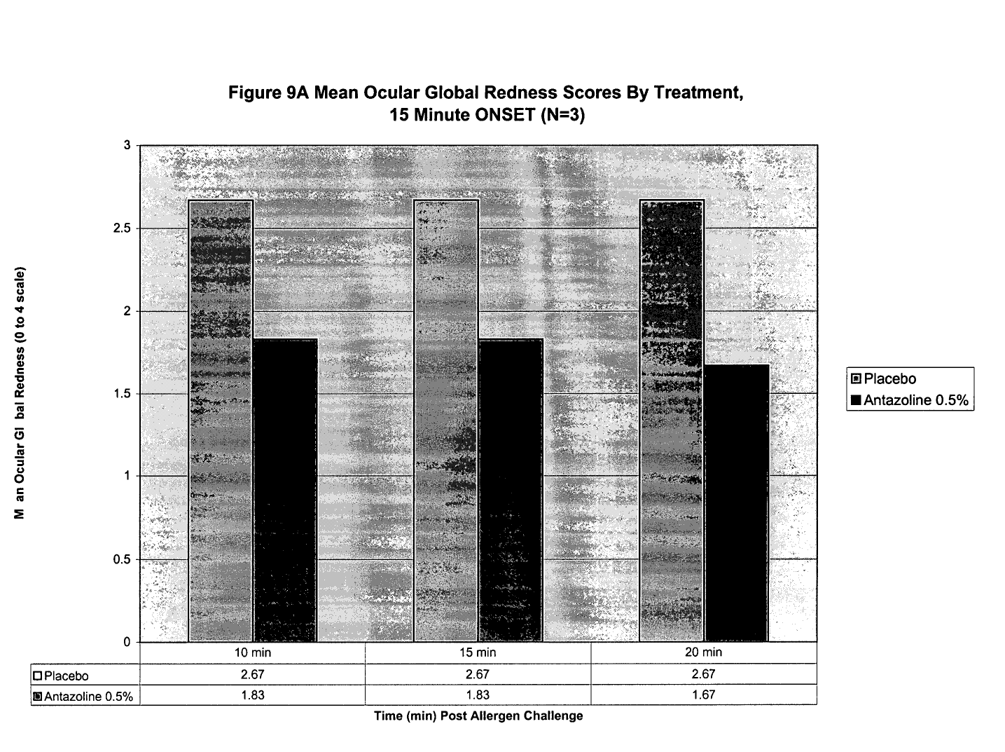

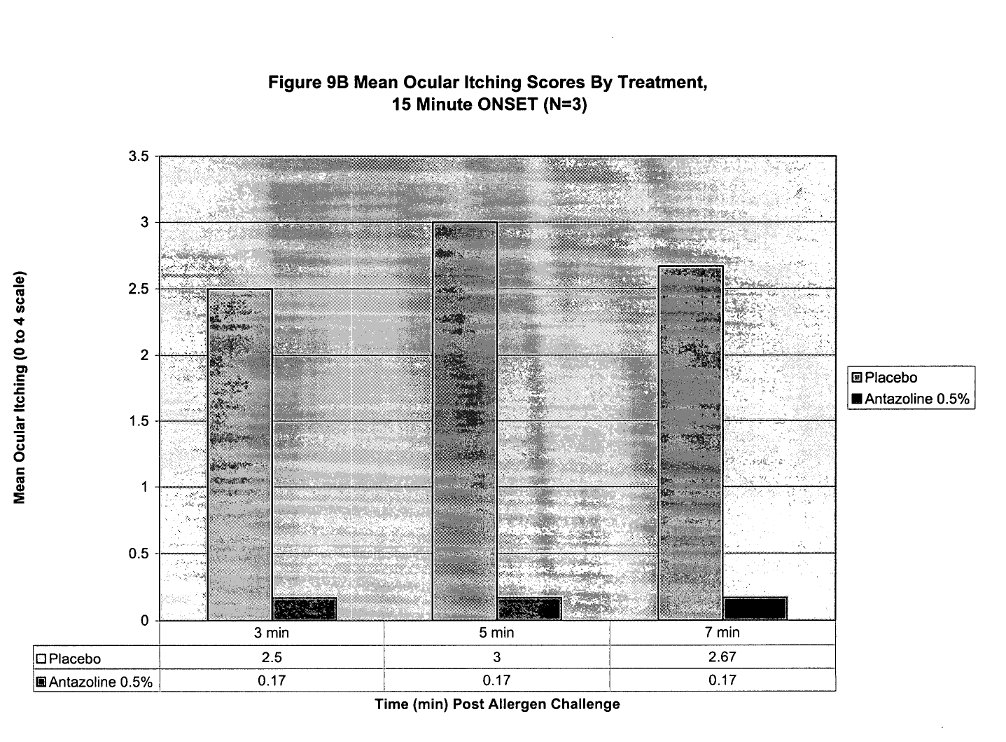

The synergistic effects of the combination of ketotifen and pheniramine is also not seen when the combination of 0.05% azelastine and 0.5% pheniramine (see FIG. 8) is tested for its efficacy in relieving both itching and redness at onset and at 4 hour duration.

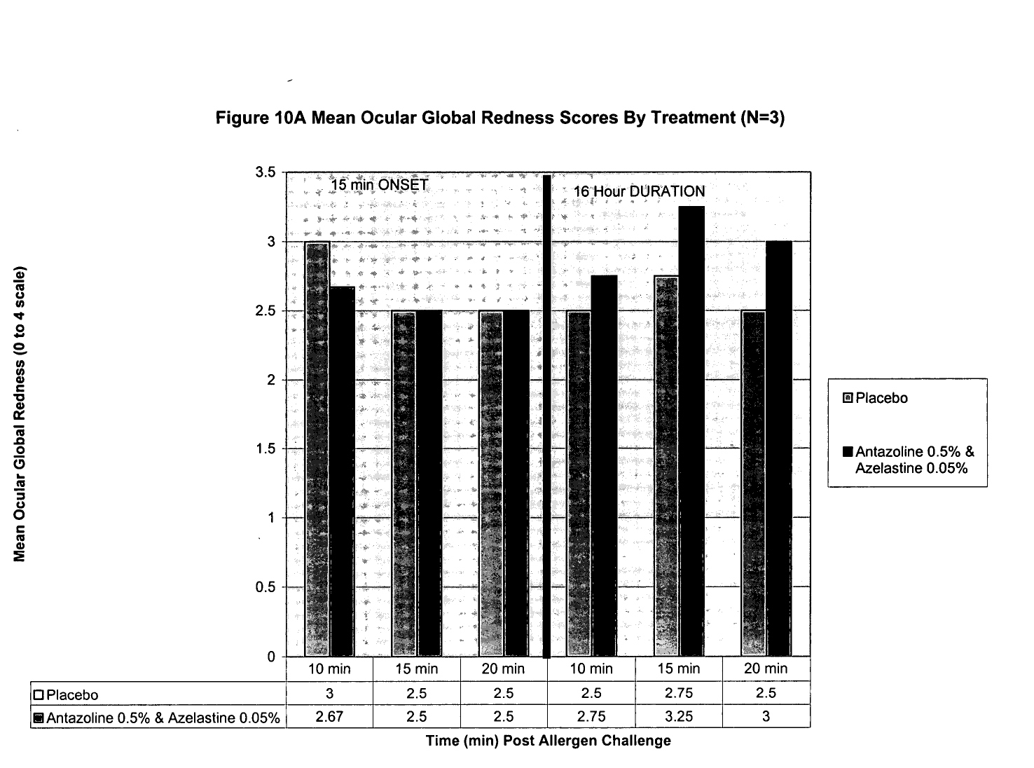

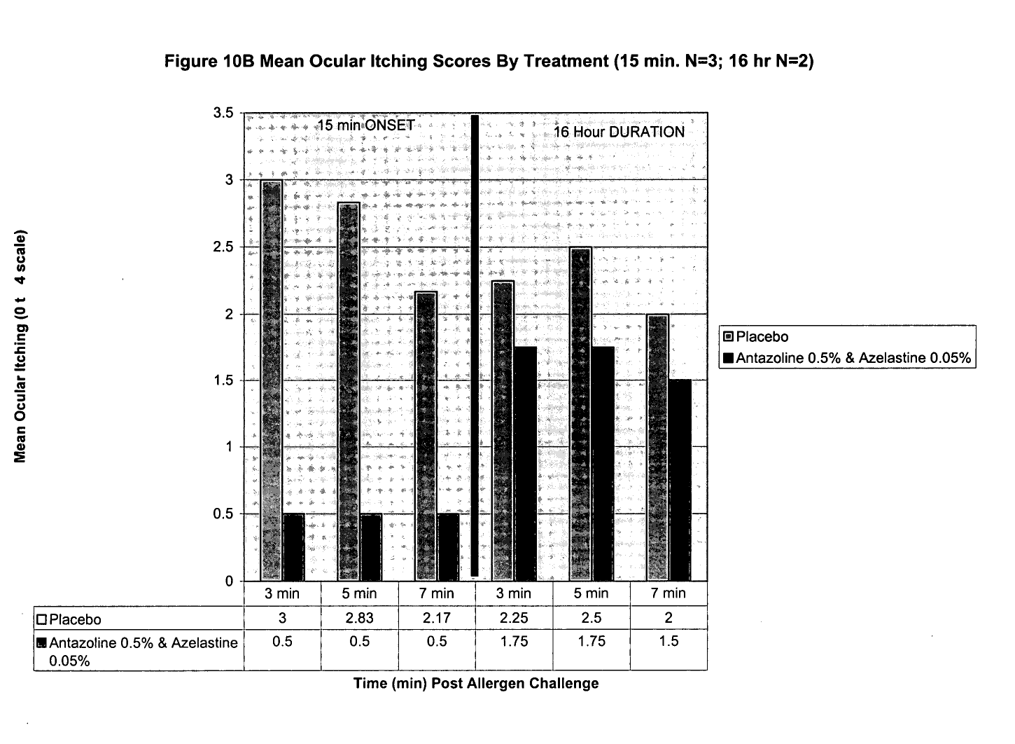

The combinational use of 0.05% azelastine and 0.5% antazoline has greater efficacy in relieving redness at 16 hour duration (see FIG. 10).

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating an ocular allergy in a subject consisting of administering to the eye surface of the subject, an effective concentration of ketotifen and an effective concentration of pheniramine in a pharmaceutically acceptable carrier.

2. The method of claim 1 wherein the effective concentration of ketotifen is between 0.04% and 0.06%.

3. The method of claim 1 wherein the effective concentration of pheniramine is between 0.4% and 0.6%.

4. The method of claim 1 further consisting of artificial tears.

5. A pharmaceutical composition consisting of an effective concentration of ketotifen and an effective concentration of pheniramine in a pharmaceutically acceptable carrier.

6. The pharmaceutical composition of claim 5 wherein the effective concentration of ketotifen is between 0.04% and 0.06%.

7. The pharmaceutical composition of claim 5 wherein an effective concentration of pheniramine is between 0.4% and 0.6%.

8. The pharmaceutical composition of claim 5 further consisting of artificial tears.

9. A method of treating an ocular allergy in a subject consisting of administering to the eye surface of the subject, an effective concentration of azelastine and an effective concentration of antazoline in a pharmaceutically acceptable carrier.

10. The method of claim 9 wherein the effective concentration of azelastine is between 0.04% and 0.06%.

11. The method of claim 9 wherein the effective concentration of antazoline is between 0.4% and 0.6%.

12. The method of claim 9 further consisting of artificial tears.

13. A pharmaceutical composition consisting of an effective concentration of azelastine and an effective concentration of antazoline in a pharmaceutically acceptable carrier.

14. The pharmaceutical composition of claim 13 wherein an effective concentration of azelastine is between 0.04% and 0.06%.

15. The pharmaceutical composition of claim 13 wherein an effective concentration of antazoline is between 0.4% and 0.6%.

16. The pharmaceutical composition of claim 13 further consisting of artificial tears.

17. A pharmaceutical composition comprising an effective concentration of azelastine and an effective concentration of a short-acting anti-histamine agent.

18. A pharmaceutical composition comprising an effective concentration of ketotifen and an effective concentration of a short-acting anti-histamine agent.

19. A pharmaceutical composition comprising an effective concentration of pheniramine and an effective concentration of a long-acting anti-histamine agent.

20. A pharmaceutical composition comprising an effective concentration of antazoline and an effective concentration of a long-acting anti-histamine agent.
